(19) United States
(12) Patent Application Publication
(10) Pub. No.: US 2004/0096369 A1
(43) Pub. Date: May 20, 2004

(54) Dispensing device

(76) Inventor: Bie Daoting, Road Guangzhou (CN)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(21) Appl. No.: 10/371,994

(22) Filed: Feb. 21, 2003

(30) Foreign Application Priority Data
Nov. 15, 2002 (CN) 02249533.9

(51) Int. Cl. A61L 9/03

(52) U.S. Cl. 422/124; 422/125; 422/62

(57) ABSTRACT

A device for blowing a vapor-containing air stream into a pre-selected space that has a housing having a channel leading to an outlet and a container for liquid mounted in the housing. A heated vaporization chamber in the housing communicates with said a channel. A nozzle sprays a fine mist of a liquid into the vaporization chamber. A blower in the channel blows a stream of air through said channel and out the outlet. A controller senses the temperature of the heater and maintains the blower and nozzle inactive until the temperature sensed reaches a predetermined level. The rate of liquid fed to the nozzle is controlled.

### BACKGROUND OF THE INVENTION

1. Field of the Invention

This invention relates to a dispensing device that actively blows an air stream containing a vaporized material, such as, a deodorizer, a fragrance, an oil, a medicine or the like, into a defined space.

2. Prior Art

It is known to dispense air fresheners and fragrances by soaking such fragrances into a gel, paper, or other absorbent substrate and permit passive evaporation of the fragrance therefrom. Such devices require the fragrance/air freshener to be volatile at ambient temperatures. Typical among these are the air fresheners typically hung from the review mirrors of cars. These are passive systems.

It is also known to heat such substrates to more rapidly release the fragrance or permit release of fragrances not normally volatile at room temperature, such as are described in Atalla et al., U.S. Pat. No. 4,816,973, issued Mar. 28, 1989, for a PORTABLE NIGHT LIGHT AND AIR FRESHENER and Wefler et al., U.S. Pat. No. 6,123,935, issued Sep. 26, 2000, for an AIR FRESHENER DISPENSER DEVICE WITH DISPOSABLE HEAT-ACTIVATED CARTRIDGE, and a myriad other issued patents too numerous to mention. These units are often adapted to plug into household wall sockets or automobile cigarette lighter sockets wherein a disposable cartridge containing fragrance-soaked substrate is inserted into the unit and replaced when the finite quantity of fragrance they contain is exhausted.

It is known to dispense atomized liquid air freshener directly into a flow of air being heated and moved by a furnace, such as is described in Davis, G. D., U.S. Pat. No. 6,435,419, issued Aug. 20, 2002, for a LIQUID AIR FRESHENER DISPENSING DEVICE FOR A DUCT. It is also known to inject atomized liquid fragrance directly into a stream of air in the context of an automotive DC system, such as is described in Shropshire, M. C., U.S. Pat. No. 5,882,256, issued Mar. 16, 1999, for a FRAGRANCE DISPENSER.

It is also known to simply blow air directly upon a substrate containing a fragrance/air freshener so as to promote evaporation of the fragrance therefrom, such as is described in Rees, N., U.S. Pat. No. 6,254,823, issued Jul. 3, 2001, for an AIR FRESHENER.

What appears to be lacking in the art is a self-contained dispensing device that can be readily connected to a power supply and that can be easily located in proximity to a space, an area, person or an article(s) that one desires to freshen or otherwise treat.

### SUMMARY OF THE INVENTION

Accordingly, it is a principal object of the present invention to provide a simple self-contained dispensing device such as a vaporizer unit that can be easily carried from place to place, set on a supporting surface, such as table, plugged into an electrical outlet in a room or space in a building, such as a home or office, and set to function in one of a plurality of modes to freshen, fumigate, or medicate the space, a pre-selected volume of air or articles located in proximity to the dispensing device. For example, the unit can easily be located in a closet and used to remove odors from clothing. Further it can also be used to freshen the air in a room such a kitchen including the walls, ceiling, furniture and appliances. Other and further details of the invention will become readily apparent from the following detailed description of preferred embodiments when taken in conjunction with the appended drawings.

### BRIEF DESCRIPTION OF THE DRAWINGS

FIG. 1 shows schematically a novel embodiment of the invention as would be suitable for use in a home, office or hospital.

FIG. 2 shows a schematically another novel embodiment of the invention suitable for use in vehicle, land or water based.

FIG. 3 shows an electrical logic diagram of the embodiment shown in FIGS. 1 and 5.

FIG. 4 shows an electrical logic diagram of the embodiment shown in FIGS. 2 and 6.

FIG. 5 shows a circuit diagram of the embodiment of the invention shown in FIG. 1.

FIG. 6 shows a circuit diagram of the embodiment of the invention shown in FIG. 2.

FIG. 7 shows the best mode in the form of a second preferred embodiment of the invention.

FIG. 8 is a medial sectional view of the preferred embodiment of FIG. 7.

### DETAILED DESCRIPTION OF THE PREFERRED EMBODIMENTS

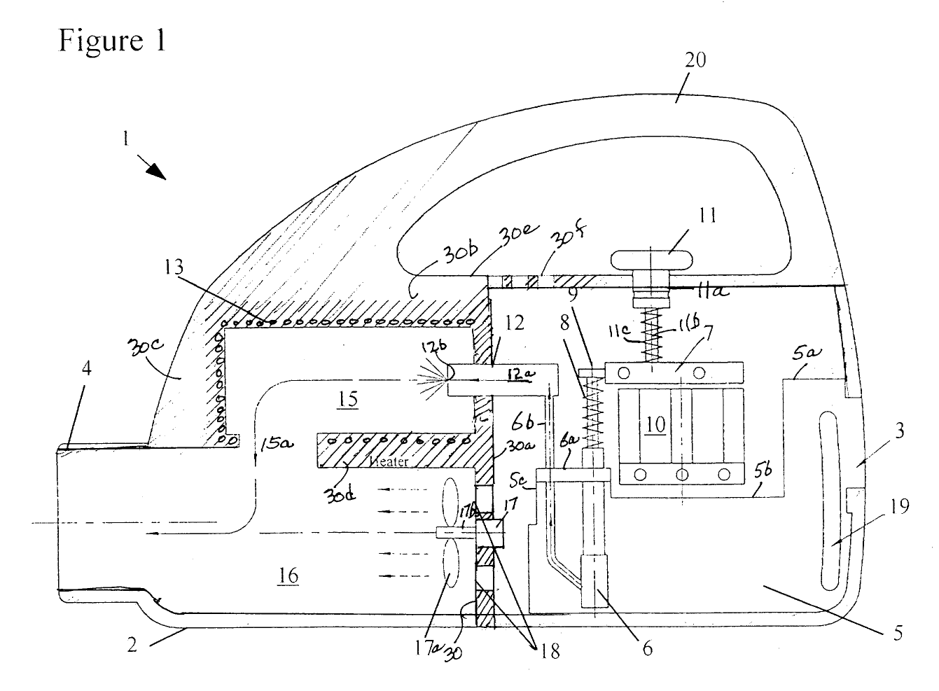

Referring to FIGS. 1, 3 and 5, there is shown a first preferred embodiment of the vaporizer device of the invention. The device generally noted as 1 consists of a main body or housing H, formed of a suitable plastic material, in the shape of a cylinder with a blunt front end and flattened rear end, and has a flat bottom 2 for sitting on a surface such as a tabletop. A handle 20 is integrally fashioned at the top of the device 1 to enable easy carrying from one location to another. The size and general shape of the device is similar to that of a conventional steam iron as available today. A liquid inlet 3 is present in the rear end surface of housing H and is closed by a cover (not shown) that can be easily opened and closed and which may be hinged or connected (but not necessarily) to the housing H of the device for this purpose. Inlet 3 leads into a container 5 mounted in the body or housing H for holding the liquid material to be dispensed by the device. Container 5 extends from the rear end to about the middle of the housing H. The rear end portion 5a of the container 5 has a depth about half the depth of the housing H and extends upwardly for about twice the depth of the main portion 5b of container 5. In rear end 5a is a sight glass 19 to enable viewing of the level of liquid in container 5a. Sight glass 19 is visible through a suitable opening in the housing H. The forward end portion 5c of the container 5 also extends above the main portion 5b, but only slightly, in order to accommodate a pump 6 and to provide a mounting plate 6a for the pump and to act as a removable closure for forward end portion 5c to enable access.

Mounted in the housing H just above the main portion 5c of the container 5 is an electromagnetic generator consisting of a core and driving coil 10 with an armature 7. This is a conventional unit is known in the art and functions when operated to drive the armature 7 up and down in a reciprocating fashion. A control knob 11 is rotatable mounted in the housing H so that upon rotation, it will move up or down depending on the direction of rotation. This is readily accomplished by a threaded connection between the housing H and the shaft or boss 11a of the knob 11. Alternatively, knob 11 can be adjusted up or down using axial vertical movement in a bore in the housing H and detents and recesses used to set the vertical position of the knob 11. The top end of a stub shaft 11b is slidably inserted in a bore formed in the free end of the shaft or boss 11a of the knob 11 and the lower other end of shaft 11b is connected to armature 7. The interaction of the top of the stub shaft 11b and the top end of the bore formed in the shaft or boss 11a provides a stop to limit the armature upward travel, thereby setting the limits of its reciprocal travel or stroke. Thus, setting the vertical position of the knob 11 in turn sets the stroke of the armature 7 and provides a flow control. Tension spring 11c is connected at one end to knob 11 and connected at its other end to armature 7 to hold the armature 7 in repose in its upper position.

A bar 9 is connected and fixed to the armature 7 to move with it. Bar 9 bears on the top end of a shaft 8 that is connected to the driving shaft of a reciprocal pump 6 that is positioned in front portion 5c of the container 5 near its bottom. The inlet for the pump 6 is not shown, but will be from the bottom area of the pump 6 in conventional fashion. The outlet from the pump 6 is via conduit 6b, as shown by the arrow in FIG. 1, which leads to the rear end of a cylindrical nozzle member 12 consisting of a reservoir 12a and a spray nozzle 12b formed in the front wall of the nozzle member 12 at its forward end. The plate closure 6a supports the reciprocating pump 6 and hold the conduit 6b in a properly oriented position. Nozzle member 12 is mounted in a support wall 30a of the housing H.

The forward end of the housing H is shaped to define a spout or outlet 4. An intermediate support wall 30 divides the front section of the housing H from the rear section of the housing H. Intermediate wall 30 consists of a vertical support wall portion 30a that is connected to the bottom 2 of the housing H and extends upwardly terminating at or near the top of the housing H, and a horizontal portion 30b that extends from the top of vertical portion 30a forwardly toward the front of housing H and then downwardly to form the front end 30c of the housing H. A wall portion 30d extends horizontally from the midpoint of vertical wall portion 30a. Wall portions 30b and 30d define a cylindrical chamber 15 into which nozzle member 12 projects with nozzle 12b pointing coaxially into cylindrical chamber 15. Front end portion 30c closes the front end of the cylindrical chamber 15 and wall portion 30a closes the rear end of the cylindrical chamber 15. Horizontal wall portion 30d terminates short of front end wall portion 30c defining a horizontally oriented opening 15a. The bottom 2, the wall portion 30a and the horizontal wall portion 30d define a second cylindrical chamber 16 that is essentially in horizontal alignment with the spout or outlet 4. Opening 15a enables communication between chamber 15 and chamber 16.

An electric dc motor 17 is supported by wall portion 30a and drives a fan 17a via its motor shaft 17b. The electrical connections and supply for the motor are not shown in FIG. 1, but will become apparent from the following description. Holes 18 in wall portion 30a enable air on the rear side of wall portion 30a to pass through the wall portion 30a to reach the fan 17a. The top 30e of the housing is provided with a grill 30f that allows air to enter the space behind or to the rear of wall portion 30a, and thereby reach the openings 18 and fan 17a. A heater 13, preferably a resistance heater, is embedded in the wall portions 30b, 30c, and 30d that surround and define the cylindrical chamber 15, in order to heat chamber 15, as will become more apparent from the following description.

The electrical circuit for the device shown in FIG. 1 is schematically shown in FIG. 5. The components to be described in conjunction with FIG. 5 are mounted in the device 1 in a manner that will be apparent to one skilled in the art from the description above taken with the description that now follows. The circuit consists of terminals 50 and 52 that are connected to a line cord provided with a conventional plug for attachment to a 110 volt electrical convenience outlet, of the type found in a home or office. Terminal 50 is connected via a fuse F1 to terminal 54 of a three way switch S1, the contacting connecting terminals of which are identified as “Timing”, “OFF” and “Manual”. Timing terminal is connected by lead 56 to a first switch S2 that is under the control of a first timer T1, and then by lead 58 to a second switch S3 under the control of a second timer T2. Lead 60 connects the switch S3 to an input terminal 62 of heater 13, the output terminal 64 of heater 13 being connected to a switch S4 that is a thermocouple Tc1 controlled switch. Lead 63 connects switch S4 to terminal 52. The Manual terminal is connected by lead 70 to a manually operated on-off switch S6 that is mounted on the housing H in a convenient position for operation by the user. Lead 72 connects switch S6 to one side of the electromagnetic generator (M1) 10, and lead 74 connects the other side of generator 10 to switch S5 that is a thermocouple Tc2 controlled switch, which in turn is connected by lead 76 to lead 63 at junction 77. The ac voltage is tapped off the electromagnetic generator 10 and fed to across a full wave rectifier D1 consisting of 4 diodes arranged in conventional fashion in a rectifying bridge. The takeoff from the rectifier bridge is a dc voltage that is connected via leads 78 and 80 across a dc motor (M2) 17 used to drive the fan 17a. Lead 82 connects a junction 62 in lead 60 with a junction 71 in lead 70. Lead 84 connects a junction 73 in lead 72 with a junction 57 in lead 56. A lead 51 is connected across terminal 50 and lead 63 and contains a resistor R1 and LED L1 in series. Similarly, a lead 86 is connected across the thermocouple operated switch S4 connecting junction 64 and lead 63, and contains in series a resistor R2 and an LED L2.

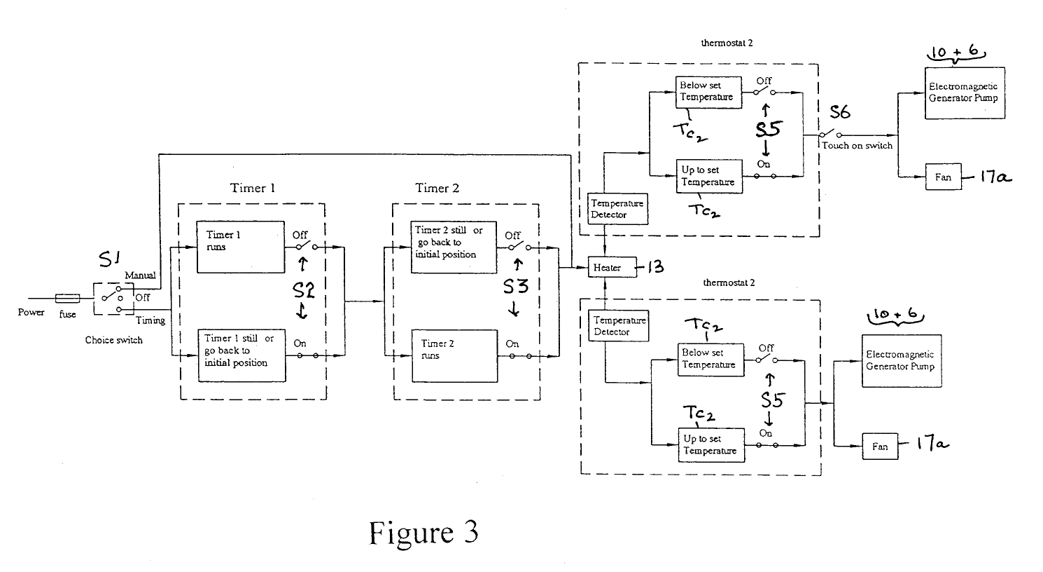

The operation of the dispensing device described above is as follows, taking into account the logic electrical working diagram of FIG. 3. The essential operation is that the chamber 15 is heated to an appropriate temperature, preferable about 100° C., before any liquid is sprayed through nozzle 12a into chamber 15. This causes the liquid mist, resulting from the spraying, to vaporize completely within the chamber 15. Meanwhile the fan 17a is being driven blowing air out the spout 4 into the space in which the device is located. This produces a slight negative pressure in chamber 16 and causes the vaporized material in chamber 15 to be drawn down through the opening 15a and diffused in and mixed into the blowing air stream being directed out the spout 4. This effect produces a much better and more efficacious distribution of the sprayed liquid material, since it is vaporized completely in heated chamber 15 and then mixed with the air blown by fan 17a. The control of the dispensing device produces this efficient operation.

The nozzle or misting mouth 12a sprays the dispensing liquid, e.g. a fragrance, as a fine mist (droplets) into the cylinder-type fuming furnace, cylindrical chamber 15 with heater 13. All of the sprayed droplets or mist is converted into vapor in the heated chamber 15. Then, the completely fumed or vaporized liquid is drawn out of heated chamber 15 into chamber 16 where it is mixed and diffused into the air being blown by fan 17a out through the spout 4 and into the designated space.

The fragrance liquid is pumped in high speed runs through the misting mouth or nozzle 12a, which has a very small diameter or series of small holes to change the pumped liquid into a fine mist. Spraying into and forming the mist in the high temperature fuming furnace produces an immediate fuming or vaporizing. All sprayed liquid is converted to vapor in chamber 15. The design of the cylindrical type of fuming furnace guarantees that the sprayed in mist doesn't come out of the device as a mist, and the fuming is 100%.

The air stream blown by and from the fan 17a travels below the furnace outlet 15a, and forms a negative pressure that speeds up vaporized material coming or flowing out of fuming furnace 15 and going out of device through spout 4. Other functions of fan 17a include strengthen air circulation in a predefined space to make the dispensed material well distributed and stronger in the air, forcing out ambient air from a predefined space and filling the space with a stronger air flow containing an appropriate amount of dispensed vaporized liquid in such space, and making a vaporized strike on a focus area which is hard to reach.

The device electrical working logic is shown schematically in FIG. 3 and is to be considered in conjunction with the electrical circuitry as shown and described with reference to FIG. 5. The working and control logic of the device will now be discussed. With respect to the working order of the functional components comprising the electrical heater 13, the electromagnetic generator 10 and fan 17a, the logic is that the heater 13 start working first, and then, the electromagnetic generator 10 and fan 17a start working at the same time after the temperature of heater 13 is up to the operating or set temperature of 100° C., that is, when liquid mist sprayed into chamber 15 will be vaporized immediately. This task is carried out by thermostat switch S5, controlled by thermocouple Tc₂, which is “on” when the temperature of heater 13 is up to the set temperature, and is “off’ when the temperature of heater is below the set temperature. The purposes of such logic and implementation are to avoid spraying mist into the fuming furnace 15 when the fuming furnace can't fume mist, to make the device intelligent, and to avoid time uncertainty when a user manually operates the device and in particular, the electromagnetic generator 10 and fan 17a.

The combination of a power switch and two timers provides working way choices for user. There are three modes of operation, namely, manual, immediate automatic dispensing, and delayed dispensing. The timer 1 is a delay. The habitus or normal condition of the switch S2 associated with and operated by timer 1 is in the “ON” state. The power is connected through S2 to the switch S3 associated with and operated by timer 2 whenever the timer 1 is still (at rest in its initial position) or goes back to its initial position. The habitus or normal condition of the switch S3 associated with and operated by timer 2 is in the “OFF” state. The power is connected to the heater 13 (H1) when the timer 2 is running (in the “ON” state), and power is cut off by switch S3 when timer 2 go back to initial position (the “OFF” state). So the combination of power switch S6 and the two timers and switches S2 and S3 provide working ways of choice.

The first mode is manual operation wherein the choice switch S1 is set to “Manual”. In this mode, heater H1 starts immediately to warm up. When the temperature sensed by Tc1 and Tc2 reach their set temperatures, switch S4 is off and indicating bulb L2 becomes red, at the same time switch S5 has switched on. Set temperature of Tc2 is lower than that of Tc1, which means that M1 and M2 are ready to work. When the power switch S6 is switched on, after the dispenser device has had a warm-up period of about 2 minutes for the heater 13 to reach operating temperature as described, M1 and M2 operate to shoot fragrance vapor and, the device starts working and dispensing a stream of air containing the vaporized liquid contained in the container 5 and being pumped through nozzle 12a. The power switch S6 can be switched on and off at any time to start and stop the further operation of the device, at the user's option. Turning switch S1 to the “OFF” position will cancel the “Manual” operation.

The second mode is no time delay with emission of the heated air stream containing the vaporized liquid for a time to be fixed or preset. The switch S1 is set to “Timing”. In this mode, the timer 1 is kept in its initial condition, still and timer 2 is set for a predetermined time that the user wants the device to operate for. The device will start working as soon as the heater reaches its operating temperature (about 2 minutes) and will issue an air stream containing the vaporized liquid continuously. The device by itself will stop operating automatically when timer 2 completes the set time. The time of issuing the air stream containing the vaporized liquid will equal the time set for timer 2 less the time required for the heater 13 to reach operating temperature (about 2 minutes).

The third mode is a set time delay and then continuous issuance of the air stream with the contained vaporized liquid for a set time. In this mode the choice switch S1 is set to “Timing”, the timer 1 is set to time T1 and timer 2 is set to a longer time T2 than T1. The machine itself will automatically start working and issuing an air stream containing the vaporized liquid after timer 1 has completed its set time, and will itself automatically stop operating after timer 2 has completed its set time. The air stream issuance time equals T2 minus T1 minus the time required for the temperature of heater 13 to rise to its operating temperature (about 2 minutes).

Because the predetermined time required for the device to reach the operating temperature of 100° C. for the heater 13 is a known factor, i.e. certain, 2 minutes, a user can take this factor into account by a very simple calculation when setting the device for one of the modes of operation as described above. A user can work the device on site or not on site to deodorize, medicate or otherwise treat an area, such as at his/her room, and/or operate the device at random or in quantization.

The thermocouple operated switch S4, controlled by thermocouple Tc₁, switches off whenever the heater temperature reaches 200° C. in order to protect the device from overheating. When the temperature of heater H1 is over about set temperature (about 200 degrees C.) of Tc1, switch S4 switches off. The current through heater H1, R2 (high resistance) and L2 become smaller, and so temperature of H1 comes down. Vice versa, when the temperature of heater H1 comes down too much, Tc1 senses such lower temperature and switch S4 is turned on to increase current and temperature of heater H1. Switch S4 is a safety mechanism. Both thermocouples Tc1 and Tc2 sense the temperature of heater 13 and are placed in the device in appropriate locations for this purpose.

FIG. 3 is a logic diagram showing schematically what has been described above. As shown Timer 1 and timer 2 and the switches S2 and S3 operated thereby are cascaded after the choice switch S1. Their outputs from a logic sense are fed to the heater where the thermocouples sense the temperature of the heater and set the switches S4 and S5 as shown in the right part of the diagram in order to control the electromagnetic generator and pump and fan. The logic diagram is set for the choice “Manual”, but is equally understandable for a setting of “Timing”.

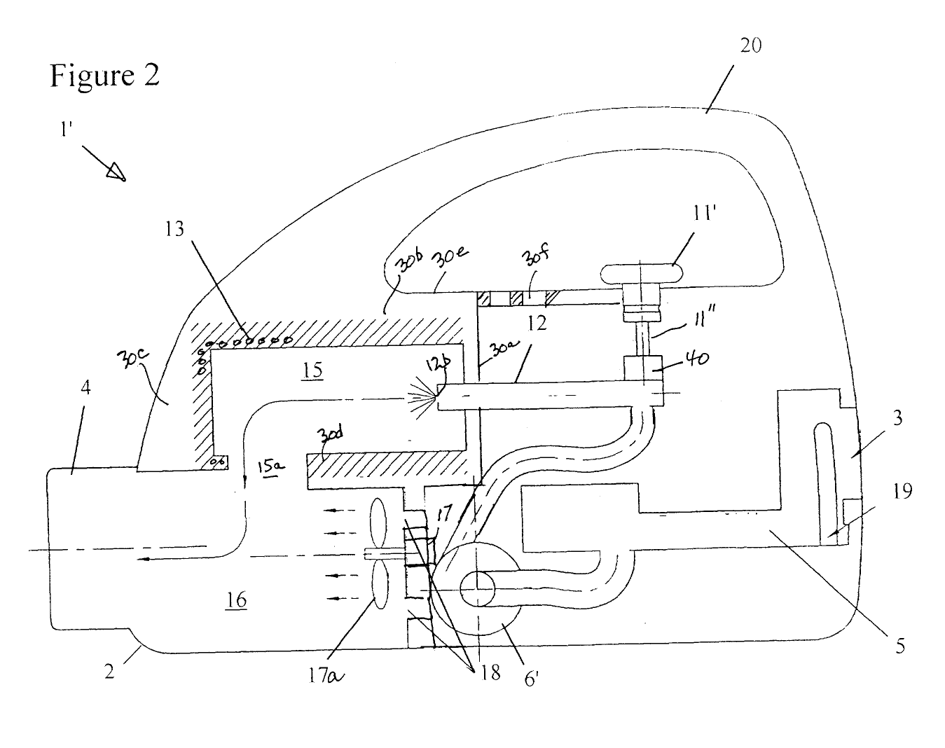

Referring now to FIGS. 2, 4 and 6, an embodiment of the invention suitable for use in an automobile or other type of vehicle, land or water, will now be described. As shown in FIG. 2, the device is similar to the device illustrated and described with reference to FIG. 1, and the same parts have been designated by the same reference numerals. In the device shown in FIG. 2, the nozzle 12a and the reservoir 12 are fed by a centrifugal pump 6′ that is coupled to the container 5 by conduit 6″. The output of pump 6′ is connected by conduit to a fitting 40 that is connected to the intake of the reservoir 12. The knob 11′ is connected by shaft 11″ to a suitable valve (not shown) residing in the fitting 40. By appropriately manipulating the knob 11′ the valve is controlled and thereby the flow from the pump is controlled as the liquid is pumped from container 5 to reservoir 12 and nozzle 12a. The remainder of the device shown in FIG. 2 is the same physically as shown and described with reference to FIG. 1.

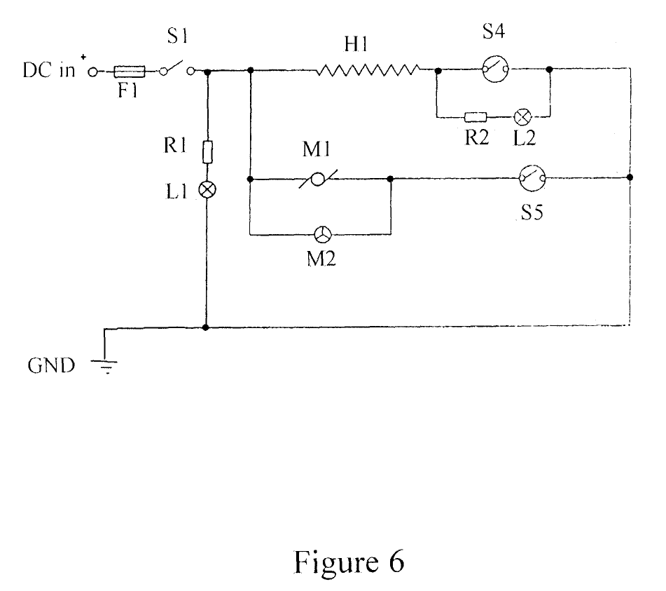

The electrical circuit for the device shown in FIG. 2 is simpler and is shown schematically in FIG. 6. A DC power source operates the circuit. Power is supplied to the B+ terminal and passes through a fuse F1 and manual switch S1 and connected to a heater electrical resistance coil or wire H1 and then through a thermocouple operated switch S4, controlled by a thermocouple Tc1, which serves as an overheating protection and then to ground GND. A high load resistor R2 and LED L2 forms a parallel path to the switch S4. On the lead in 100 to the heater H1, is a junction connected to ground via a high load resistor R1 and LED L1. From junction 104, also in the lead 100 to the heater H1, is connected a lead 106 that connects in parallel to the motor M1 driving the centrifugal pump 6′ and motor M2 that drives the fan 17a. The two motors are connected in common by junction 108 to ground through a second thermocouple operated switch S5 controlled by thermocouple Tc2.

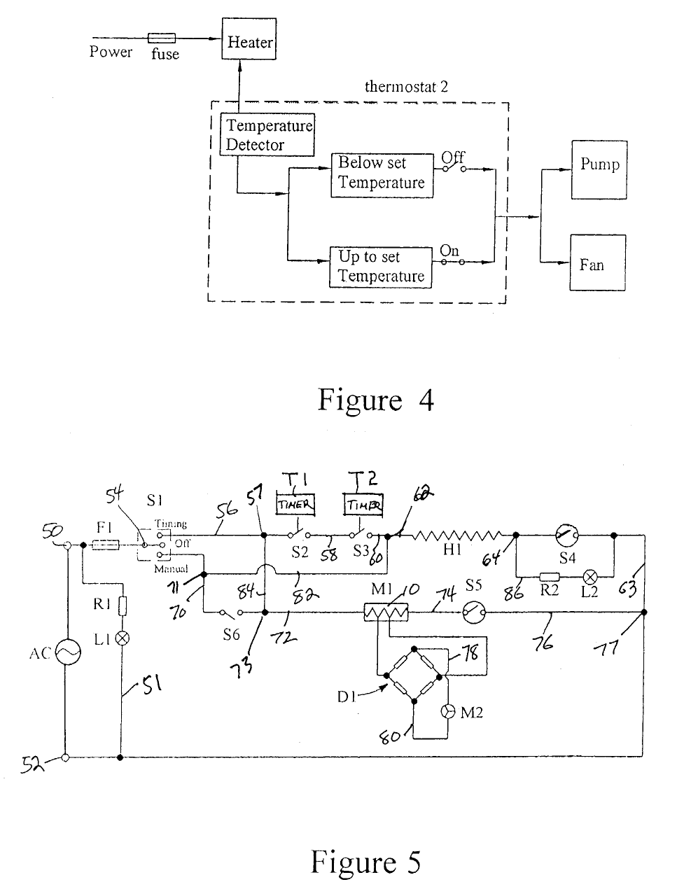

As will be apparent from the Logic electrical working diagram shown as FIG. 4, the operation of the device shown is FIG. 2 is quite simple. Whenever the manual switch S1 is closed, the DC current will flow through the heater H1 causing the heater H1 to start heating up, since overheating protector switch S4 will be close due to the sensing of thermocouple Tc1 indicating that the heater H1 is below the overheated state (above 200° C.). Meanwhile, the motors will not be driving, because the thermocouple Tc2 controlling the switch S5 will be sensing that the heater H1 is below the set or operating temperature (below 100° C.), and therefore, switch S5 will be OFF. When the thermocouple Tc2 senses that the temperature of heater H1 has risen to or above the set or operating temperature, switch S5 will be ON, at which time the motors M1 and M2 will be driving the pump 6′ and the fan 17a, respectively.

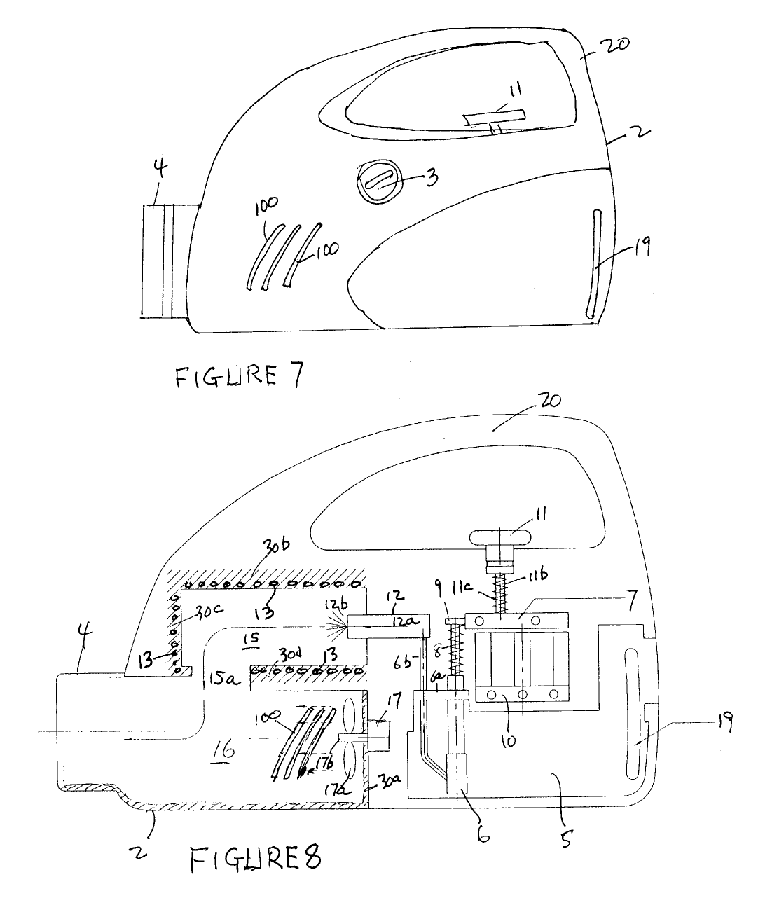

Referring now to FIGS. 7 and 8, the best mode for the housing is shown in the second preferred embodiment. Like parts have been designated by the same reference numbers. The main difference is the provision of air inlet slots 100 formed on each side of housing 2 that provide openings into chamber 16 axially spaced forward of fan 17a toward spout 4. This simplifies and enhances the capability of fan 17a drawing air into the housing 2 to be pushed out spout 4. The other difference is the relocation of the filling inlet 3 from the rear of housing 2 to side of housing 2, in a more convenient location that enables filling with greater ease and efficiency. In the location as shown, filling inlet is slightly canted upwardly.

Changes and modifications as apparent to those skilled in the art from the embodiments as disclosed herein are deemed to fall within the purview of the claims.

What is claimed is:

1. A device for blowing a vapor-containing air stream into a pre-selected space comprising:
a housing having a channel leading to an outlet;
a container for liquid mounted in said housing;
a vaporization chamber defined within said housing having a communication with said channel;
a heater for heating said vaporization chamber;
a nozzle for spraying a fine mist of a liquid into the vaporization chamber;
a liquid feeder for feeding said liquid from said container to said nozzle;
a blower mounted in said channel for blowing a stream of air through said channel and out said outlet;
a controller comprising a first control including a temperature sensor for sensing the temperature of the heater and maintaining the blower and liquid feeder inactive until the temperature sensed reaches a predetermined level, and a second control for varying the rate of liquid fed to the nozzle.

2. A device according to claim 1 wherein the controller includes a third control for protecting against overheating of the heater.

3. A device according to claim 1 wherein the controller includes a pair of timers each controlling a switch

4. A device according to claim 1 wherein the controller includes a choice switch.

5. A device according to claim 1 wherein the controller enable a plurality of modes of operation.

6. A device according to claim 1 wherein the liquid feeder includes a pump and a driving motor.

7. A device according to claim 1 wherein the blower includes a fan and a driving motor.

8. A device according to claim 6 wherein the pump is a reciprocating pump and the driving motor is an ac motor.

9. A device according to claim 6 wherein the pump is a centrifugal pump and the driving motor is a dc motor.

10. A device according to claim 1 wherein the vaporization chamber is located directly above the channel and vertically separated therefrom by a common horizontally extending wall portion and an opening is defined in the common wall portion spaced from the nozzle establishing communication between the vaporization chamber and the channel.

11. A device according to claim 1 wherein the heater includes a resistance wire embedded in the wall portions defining the vaporization chamber.
